(19) United States
(12) Patent Application Publication
(10) Pub. No.: US 2012/0258444 A1
(43) Pub. Date: Oct. 11, 2012

(54) ACOUSTIC WAVE (AW) SENSING DEVICES USING LIVE CELLS

(76) Inventors: Joel M. Therrien, Westford, MA (US); Susan J. Braunhut, Wellesley, MA (US); Kenneth A. Marx, Francestown, NH (US); Gang Wang, Lowell, MA (US); Malavika Vashist, Wappingers Falls, NY (US); Abiche H. Dewilde, Tewksbury, MA (US); Jianping Zhang, Nashua, NH (US); Hongwei Sun, Lexington, MA (US); Lian Dai, Lowell, MA (US); Sai Liu, Colombia, MO (US)

(21) Appl. No.: 13/300,407

(22) Filed: Nov. 18, 2011

Related U.S. Application Data
(60) Provisional application No. 61/415,249, filed on Nov. 18, 2010.

(51) Int. Cl. G01N 29/12; C12M 1/34

(52) U.S. Cl. 435/5; 435/29; 435/287.1

(57) ABSTRACT

In one embodiment according to the invention, there is provided a method of sensing a response of a living cell or virus to a change in conditions. The method comprises applying an essentially constant external electromotive force that causes oscillation of an acoustic wave device at essentially constant amplitude and frequency under steady state conditions. The acoustic wave device has attached at least one living cell or virus. A combined oscillating system including the acoustic wave device and the living cell or virus exhibits a fundamental frequency and at least one harmonic frequency of the combined oscillating system. The living cell or virus is exposed to a change in an environmental condition while oscillating the combined oscillating system under the essentially constant external electromotive force, whereby a response of the living cell or virus to the change in environmental condition will be indicated by a change in at least one of frequency and amplitude of the oscillation of at least one harmonic frequency of the combined oscillating system.

### RELATED APPLICATION

This application claims the benefit of U.S. Provisional Application No. 61/415,249, filed on Nov. 18, 2010. The entire teachings of the above application are incorporated herein by reference.

### GOVERNMENT SUPPORT

This invention was made with government support under W911NF-09-2-0046 awarded by the U.S. Army Research Office. The government has certain rights in the invention.

### BACKGROUND OF THE INVENTION

In recent years, acoustic wave (AW) devices such as thickness shear mode resonators, i.e., quartz crystal microbalance (QCM), or shear-horizontal surface acoustic wave (SAW) devices have been used as mass measuring tools to study in real time and in liquids the kinetics of enzyme-substrate degradation, protein binding rates and as chemical sensors (1-8). The principle of these devices is that a quartz crystal oscillates between two electrodes at a frequency determined by its mass and cut. Most QCM devices use AT cut (a designation referring to the type of cut of the crystal resonator plate from the source crystal) 10 megahertz (MHz) crystals which oscillate 10 million times per second. This oscillation frequency is highly stable and the basis for the longevity and precision of quartz watches. SAW devices can operate at much higher frequencies (25 to 500 MHz) and typically use ST cut quartz crystals (9). QCM and SAW devices have much in common. Both have thin metal conducting layers on a piezoelectric crystal and can provide information about a wide variety of materials when associated with the crystal surface. Recent studies indicate SAW devices are superior to bulk wave devices (QCM) in that they are easier to make and can operate at higher, more mass sensitive frequencies. If mass is added to the crystal surface, the oscillation frequency will decrease with a sensitivity sufficient to detect deposition of a single layer of atoms.

QCMs have been modified to incorporate living cells and to measure changes in crystal oscillation frequency. Cells added to the surface of the crystal, couple their mass to it and reach equilibrium Impedance can also be measured and provides information about the visco-elastic properties of the coupled mass, and in the case of cells, reflects assembly and arrangement of the cell's cytoskeletal elements. Once cells reach a homeostatic attachment state, a new baseline of frequency is established, and if any agent is now added to living cells that causes the cells to divide, migrate, die, biotransform, differentiate or polarize, changes in frequency and impedance can be rapidly detected with an AW device. These frequency and impedance measurements can reflect internal structural cell information in important ways. However, previous acoustic wave devices typically do not provide information regarding biological responses of subcellular structures.

Therefore, there is a need for an acoustic wave device that minimizes or overcomes the above-mentioned difficulties.

### SUMMARY OF THE INVENTION

In accordance with an embodiment of the invention, there is provided an acoustic wave device with associated electronics for use in sensing the biological response of a living cell, which has increased sensitivity to the biological response by virtue of coupling into higher harmonics of the resonant frequencies of the oscillating device and cell.

In one embodiment according to the invention, there is provided a method of sensing a response of a living cell or virus to a change in conditions. The method comprises applying an essentially constant external electromotive force that causes oscillation of an acoustic wave device at essentially constant amplitude and frequency under steady state conditions. The acoustic wave device has attached at least one living cell or virus. A combined oscillating system including the acoustic wave device and the living cell or virus exhibits a fundamental frequency and at least one harmonic frequency of the combined oscillating system in response to the electromotive force. The living cell or virus is exposed to a change in an environmental condition while oscillating the combined oscillating system under the essentially constant external electromotive force, whereby a response of the living cell or virus to the change in environmental condition will be indicated by a change in at least one of frequency and amplitude of the oscillation of at least one harmonic frequency of the combined oscillating system.

In a further embodiment according to the invention, there is provided a method of sensing a response of a living cell or virus to a change in conditions. The method comprises band pass filtering an electrical driving signal through an acoustic wave device, the acoustic wave device having attached at least one living cell or virus, whereby a combined oscillating system including the acoustic wave device and the living cell or virus exhibits oscillation at a fundamental frequency and at least one harmonic frequency of the combined oscillating system. The cell or virus is exposed to a change in an environmental condition while band pass filtering through the acoustic wave device, whereby a response of the cell or virus to the change in conditions will be indicated by a change in at least one of the frequency and amplitude of the oscillation of at least one harmonic frequency of the combined oscillating system.

In another embodiment according to the invention, there is provided an apparatus for sensing a response of a living cell or virus to a change in conditions. The apparatus comprises an acoustic wave device and an electromotive drive connected to the acoustic wave device that causes oscillation of the acoustic wave device at a fundamental and at least one harmonic frequency. The electromotive drive includes a signal generator electrically coupled to provide an electrical driving signal that is band pass filtered through the acoustic wave device.

In another embodiment according to the invention, there is provided an apparatus for sensing a response of a living cell or virus to a change in conditions. The apparatus comprises: a) an acoustic wave device, the acoustic wave device having applied an essentially constant external electromotive force that causes oscillation of the acoustic wave device at essentially constant amplitude and frequency under steady state conditions; b) at least one living cell or virus attached to the acoustic wave device; and c) an electronic circuit connected to the acoustic wave device, the electronic circuit including: (i) an oscillator circuit oscillating at the fundamental frequency of a combined oscillating system including the acoustic wave device and the living cell or virus; and (ii) at least one multiplier circuit oscillating at one or more harmonic frequencies of the combined oscillating system.

In a further embodiment according to the invention, there is provided a method of sensing a response of a living cell or virus to a change in conditions. The method comprises the steps of: a) applying an essentially constant external electromotive force that causes oscillation of an acoustic wave device at essentially constant amplitude and frequency under steady state conditions, the acoustic wave device having attached at least one living cell or virus, whereby a combined oscillating system including the acoustic wave device and the living cell or virus exhibits a fundamental frequency and at least one harmonic frequency of the combined oscillating system; and b) exposing the living cell or virus to a change in an environmental condition while oscillating the combined oscillating system under the essentially constant external electromotive force, whereby a response of the living cell or virus to the change in environmental condition will be indicated by a change in at least one of frequency and amplitude of the oscillation of at least one harmonic frequency of the combined oscillating system. The essentially constant external electromotive force is applied by an electronic circuit connected to the acoustic wave device, the electronic circuit including: (i) an oscillator circuit oscillating at the fundamental frequency of a combined oscillating system including the acoustic wave device and the living cell or virus; and (ii) at least one multiplier circuit oscillating at one or more harmonic frequencies of the combined oscillating system.

In another embodiment according to the invention, there is provided a method of sensing a response of a molecularly-imprinted polymer to a change in conditions, the method comprising the steps of: a) applying an essentially constant external electromotive force that causes oscillation of an acoustic wave device at essentially constant amplitude and frequency under steady state conditions, the acoustic wave device having attached at least one molecularly-imprinted polymer, whereby a combined oscillating system including the acoustic wave device and the molecularly-imprinted polymer exhibits a fundamental frequency and at least one harmonic frequency of the combined oscillating system; and b) exposing the molecularly-imprinted polymer to a change in an environmental condition while oscillating the combined oscillating system under the essentially constant external electromotive force, whereby a response of the molecularly-imprinted polymer to the change in environmental condition will be indicated by a change in at least one of frequency and amplitude of the oscillation of at least one harmonic frequency of the combined oscillating system. The essentially constant external electromotive force is applied by band pass filtering the output of a white noise generator through the acoustic wave device.

An embodiment according to the invention has the advantage of permitting coupling of an acoustic wave device with the resonant frequencies of a living cell or virus, near which waves passing through the living cell or virus will be more strongly affected by the viscoelastic properties of the living cell or virus. Another advantage of an embodiment according to the invention is that it can provide information about the viscoelastic properties of the coupled mass, and in the case of cells, can reflect assembly and arrangement of the cell's cytoskeletal elements and other internal structural cell information.

If any agent is added to the living cells coupled to the acoustic wave device that causes the cells to divide, migrate, die or biotransform, redistribute or rearrange subcellular organelles, change shape, attachment geometry, or other alterations to the mass or viscoelastic elements within the cell, any resulting changes in frequency and impedance can be rapidly detected with the acoustic wave device. The invention can also have a compact circuit, which permits the device to be miniaturized to a high degree. In one embodiment, single circuit drives more than one harmonic simultaneously. In another embodiment, multiple oscillators can be contained in one substrate, allowing for parallel testing and increased throughput.

An embodiment according to the invention further provides the advantage of permitting specific information about sub-cellular structures to be obtained by operating the acoustic wave device at multiple harmonics, thereby reflecting the complex structure of the cell, which may have a complex frequency response. Particular frequencies may relate to specific structures within the cell. Obtaining a frequency response curve by driving an acoustic wave device at different harmonics may allow more detailed diagnostics of the status of the cells.

The invention provides advantages over other biosensor platforms for a number of reasons. Whereas traditional assays usually have to be “trained,” a live cell based sensor doesn't need to be trained, since the cells already know what is a toxin, a stimulus, an inhibitor, a differentiation agent, a carcinogen and so forth, and can serve as a proxy of the human body. Further, most traditional assays use radiolabels or fluorescent labels, whereas a cell based sensor technology is label free. Further, traditional assays usually measure single endpoints at single times, whereas a cell based acoustic wave device can accomplish continuous monitoring over days or weeks. In addition, traditional assays have endpoints that are not in real time and that take hours or days to process and analyze. By contrast, the readout from a live cell biosensor is in real time and can be automated and the output patterns can be compared to a library of response patterns.

The number of transistors and electronic components is reduced as compared to previously published circuits for driving piezo oscillators under a damping load. Further, a single oscillator is capable of driving more than one harmonic simultaneously. Multiple oscillators can be contained in one substrate, allowing for parallel testing and increased throughput, for example with a SAW-based sensor. Frequency may be optimized for maximum sensitivity to the cells. The frequency dependent response of the oscillator can be used to obtain more detailed information about the structure of the cells. In addition, the device may be small, portable, inexpensive, high throughput and operate at multiple frequencies. The associated electronics may permit remote data acquisition. Any type of living cell can be used as the sensing element.

The invention may be used for drug discovery. A known panel of human malignant cells could be used and tested to discover new chemotherapeutics as a class of drug with a specific action on the cell; for example, taxanes or epothilones which act on microtubules and have a targeted specificity of drug action. Other techniques of drug discovery, diagnosis, guiding cancer therapies, detecting infectious pathogens, studying drug transport across natural vascular permeability barriers and other techniques may be used, in similar ways to those described in U.S. Pat. No. 7,566,531 B2 of Marx et al., the entire disclosure of which is hereby incorporated herein by reference.

The invention may be used for customized therapeutics using a biopsy sample. If a patient develops resistance there is currently no way to predict which alternative therapeutic will rescue efficacy of treatment. Using a set of AW devices and a patient's tumor cells from a biopsy, the patient's tumor cells could be tested against a panel of potential alternative treatments and the one most effective selected before treatment is begun. If a patient becomes allergic to the drug, which does happen in response to many chemotherapeutics, the above method for selecting an alternative could be used.

The invention device may be used for drug discovery for virally-infected human cells, bacterial infected human cells, fungal infected human cells, protozoa infected human cells and amoebic infected human cells. Cells infected with resistant strains of the above organisms could lead to the discovery of new and more effective antimicrobial drugs. More generally, the device may be used for drug discovery for antibacterials, anti-virals, anti-fungals, anti-protozoas, anti-parasitics, anti-amoebic and anti-malignancy drugs.

The invention may be used for identification of new toxins or for environmental monitoring. Normal human cells can be used to test suspected carcinogens, toxins, pathogens, gases in the laboratory or in the work place. For environmental monitoring anything that adversely affects the human cells can be detected, for example, depletion of oxygen, such as in mines

For environmental monitoring, the invention may be used for monitoring gases, toxins, pathogens, radiation, oxygen deprivation and water safety.

The invention may also be used for customized therapy, in similar fields to those discussed above for drug discovery.

The invention may be used for toxicity testing, for example for engineered nanomaterials. Further, the device may be used to implement a safety appliance, such as a mine safety appliance.

The invention may also be used for differentiation agent testing, for example using adherent stem cells and monitoring a loss of proliferative potential and gain of cell polarity, adhesion symmetry and/or apical-basal domain definition.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a mechanical model of an acoustic device-cell system in accordance with an embodiment of the invention.

FIG. 2 is a functional block diagram of a sensor readout circuit in accordance with an embodiment of the invention.

FIGS. 3A and 3C are circuit diagrams for two possible configurations for using the acoustic wave device as a narrow band pass filter for identifying the resonant frequency, and FIGS. 3B and 3D are corresponding simulated frequency spectra, in accordance with an embodiment of the invention.

FIG. 4 is a graph of power spectrum data from an active filter using a 4.26 MHz crystal in the feedback loop, in accordance with an embodiment of the invention.

FIG. 5 is a photograph of a QCM style AW sensor module, in accordance with an embodiment of the invention.

FIG. 6 is a graph showing frequency shift results of an experiment with human mammary epithelial cells treated with growth factor, in accordance with an embodiment of the invention.

FIG. 7 is a diagram of a sensor circuit using a frequency multiplier, in accordance with an embodiment of the invention.

FIG. 8 is a diagram of a biosensor using a selective substrate film, in accordance with an embodiment of the invention.

FIG. 9 is a block diagram of a sensor configured to determine a change in complex impedance, in accordance with an embodiment of the invention.

FIG. 10 is a diagram of electrodes of a surface acoustic wave device, in accordance with an embodiment of the invention.

FIG. 11 is a graph showing a frequency response to sodium azide addition to cells on a surface acoustic wave device, and attenuation values from the surface acoustic wave device, in accordance with an embodiment of the invention.

FIG. 12 is a graph showing a frequency response from a trypsin enzymatic release experiment on cells on a surface acoustic wave device, in accordance with an embodiment of the invention.

### DETAILED DESCRIPTION OF THE INVENTION

A description of example embodiments of the invention follows. In an acoustic device-living cell system, how strongly the visco-elastic properties of the living cells affect the oscillation of the acoustic wave device will be dependent on the frequency of oscillation. At very low frequencies, the living cells will act as additional mass. However as an elastic body, a living cell can be expected to have a resonant frequency near which waves passing through the living cell will be more strongly affected by the visco-elastic properties of the living cell. If the acoustic wave device is oscillating at frequencies near this, then it will be coupling into these properties. If the cell is assumed to be spherical and have visco-elastic properties similar to water, for a cell with a diameter of 10 microns, the resonant frequency will be approximately 50 MHz. As the density and rigidity of the cell differs from water this frequency will cover a broad range, and the likely range of frequencies are easily covered by a SAW resonator. Viscosity within the cell will result in a broadening of the frequency response, but does not cause a shift in the frequency. Based on this estimate, AW based devices are very likely to span the needed frequency range to reach the optimal frequency when using live cells.

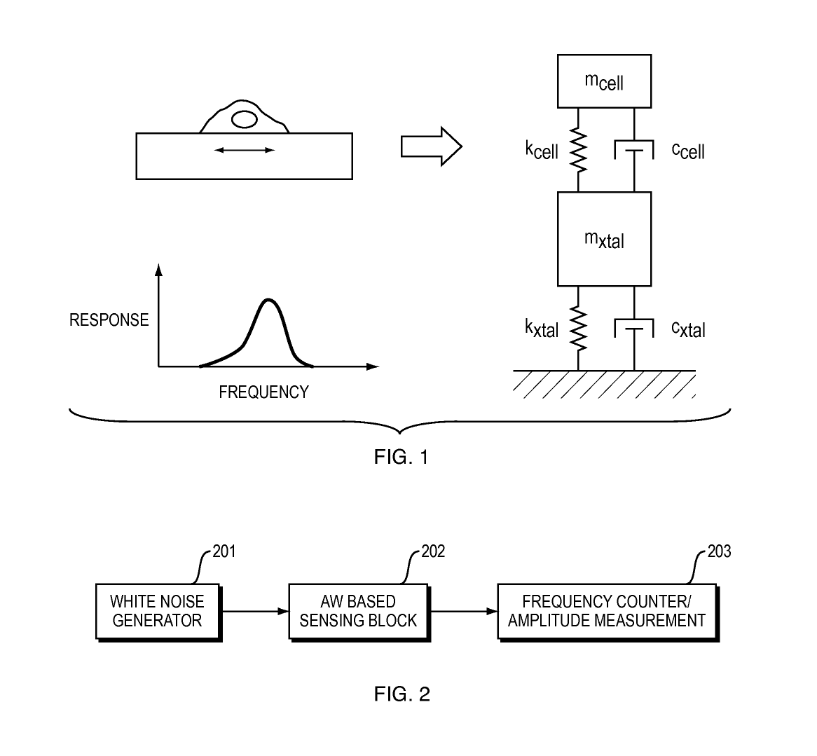

Without wishing to be bound by theory, it is believed that an acoustic device-cell system can be better mechanically modeled, as compared with conventional mechanical models of acoustic device-cell systems, by modeling the system as a mass-spring-mass-spring system, as shown in FIG. 1. The AW device of FIG. 1 is represented as a damped mass spring oscillator characterized by a mass mₓₜₐₗ, a spring constant kₓₜₐₗ and a damping constant Cₓₜₐₗ. Coupled to this is the living cell, which also is represented as a damped mass-spring system, characterized by a mass m_{cell}, spring constant k_{cell} and a damping constant C_{cell}. The complete system will have a peak response to an external electrical oscillation when the resonant frequencies of the external electrical oscillation and the complete system are matched.

An embodiment according to the invention takes advantage of the fact that cells, as mechanical systems, will have a different degree of coupling to the acoustic wave device surface depending on the frequency of oscillation. Cells behave like a visco-elastic structure, with a specific resonant frequency. The closer the acoustic wave device oscillation is to that frequency, the stronger the coupling between the cells and acoustic wave device. Thus, the acoustic wave device will have the greatest sensitivity to the cells' mechanical properties near the resonant frequency.

The invention simultaneously oscillates an acoustic wave device at the fundamental frequency as well as a higher harmonic, enabling more information about the cells to be obtained. In one embodiment, the invention oscillates the acoustic wave device at the third harmonic and other higher harmonics, for example the higher odd harmonics, such as the third, fifth, seventh and higher odd harmonics.

A circuit in accordance with an embodiment of the invention tests the admittance (how easily a signal will enter a device) at a wide range of frequencies simultaneously. A circuit in accordance with an embodiment of the invention presents all frequencies at the same time and exploits the narrow band pass of the AW device to only pass a few frequencies corresponding to resonant frequencies. With the living cell being attached to the acoustic wave device, the circuit therefore functions as a band pass filter to pass through the fundamental resonant frequency, and higher harmonics, of the combined mechanical system of the acoustic wave device plus living cell.

FIG. 2 is a functional block diagram of a sensor readout circuit in accordance with an embodiment of the invention. A white noise generator 201 provides a stimulus that is fed into a circuit 202 containing the acoustic wave sensor. The white noise generator 201 produces an input signal with a very large bandwidth. It will be appreciated that any of a variety of different devices may be used for generating the white noise. However, in accordance with an embodiment of the invention, the spectrum that the white noise generator 201 covers may be in the range of from about 1 MHz at the low end to a few tens of MHz at the upper end, or to a few GHz at the upper end, depending on the AW device being used. In circuit 202, the AW sensor acts as a band pass filter to restrict the frequencies passed to the frequency counter 203 to be the resonant frequencies of the acoustic wave sensor plus the attached living cell. The frequency and amplitude of the output of the AW sensing block 202 is then measured and recorded.

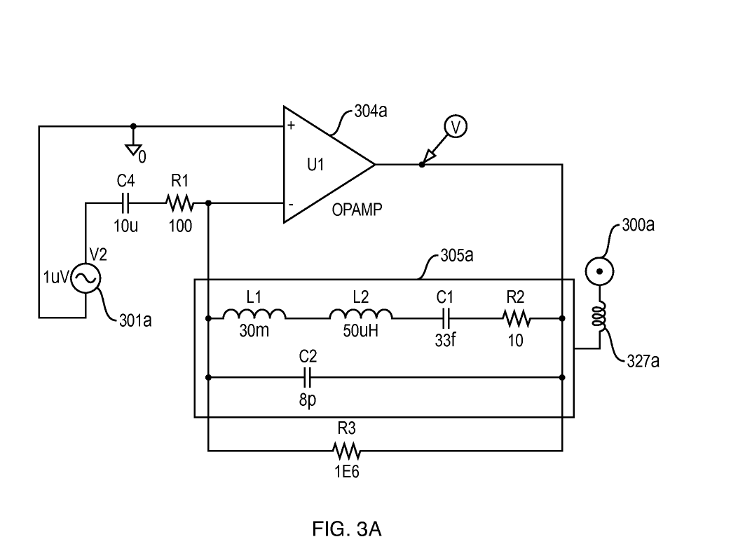

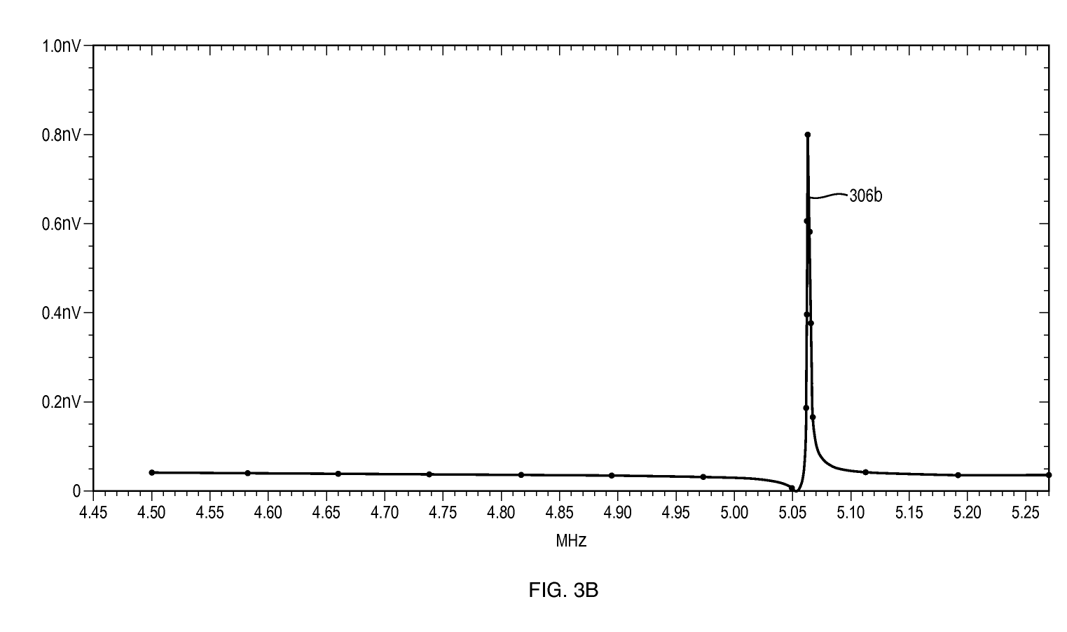

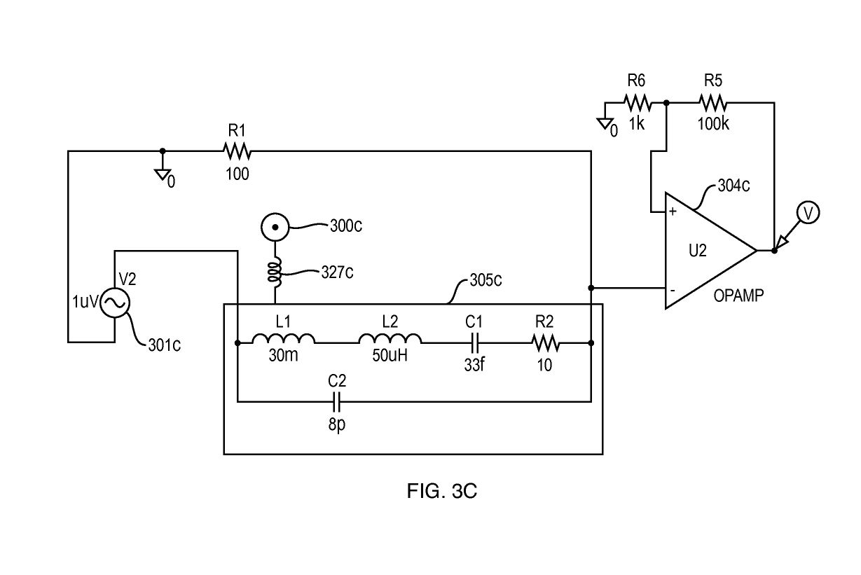

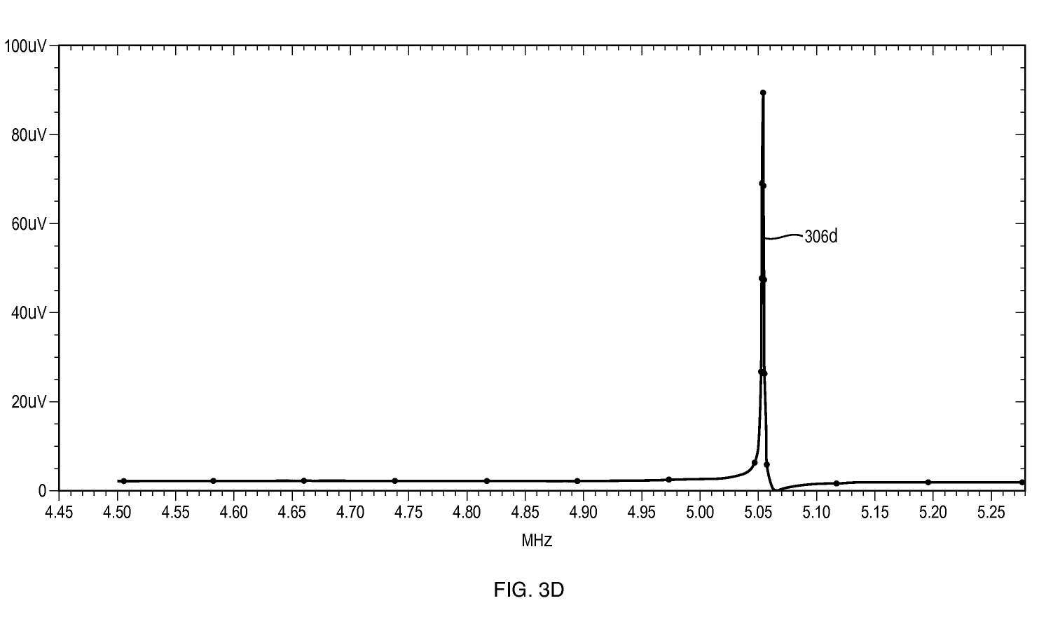

FIGS. 3A and 3C are circuit diagrams for two possible configurations of embodiments of the invention for using the acoustic wave device as a narrow band pass filter for identifying the resonant frequency, and FIGS. 3B and 3D are corresponding simulated frequency spectra exhibited by use of the invention. In FIG. 3A, the acoustic wave device is used as the tuning element in an active filter. The circuit includes a white noise generator 301a, amplifier 304a and acoustic wave device incorporating a piezo-electric material, for which an equivalent circuit is depicted within box 305a in FIG. 3A. The piezo-electric material may, for example, include a crystal, an amorphous material, a polymer, or another piezo-electric material. For example, the piezo-electric material may include one or more of: quartz, barium titanate, lithium niobate and/or polyvinyldine fluoride, and/or any of the materials described in E. Fukada, History and Recent Progress in Piezoelectric Polymers, IEEE Transactions on Ultrasonics, Ferroelectrics, and Frequency Control, Vol. 47, No. 6, November 2000, the entire disclosure of which is hereby incorporated herein by reference. The acoustic wave device is used as the feedback element to the amplifier 304a. The resulting frequency spectrum of the output, shown in FIG. 3B, shows a narrow peak 306b at the acoustic wave device's resonant frequency (˜5 MHz). Alternately, the acoustic wave device in series with a resistor may be used as a band pass filter in a passive circuit, as shown in

FIG. 3C. The circuit of FIG. 3C includes a white noise generator 301c, amplifier 304c and acoustic wave device, for which an equivalent circuit is depicted within box 305c in FIG. 3C. The output of the circuit of FIG. 3C will not have any gain and as such may require amplification before further processing takes place, for instance by inserting an amplifier between the acoustic wave sensing block (202 of FIG. 2) and the frequency counter (203 of FIG. 2). The output spectrum for the circuit of FIG. 3C can be seen in FIG. 3D, with a narrow peak 306d at the acoustic wave device's resonant frequency.

In accordance with an embodiment of the invention, if harmonics are transmitted through the AW device, a series of selectable wide band pass filters may be employed to select out frequency ranges known to correspond to specific oscillation modes. In addition to frequency, the amplitude of the measured signal will correspond to the motional impedance of the AW device.

By enabling the circuit of box 305a/305c to act as a band pass filter, which may be performed with a living cell 300a /300c attached to the circuit or acoustic wave device that embodies box 305a/305c, the circuits of FIGS. 3A-3D permit an embodiment according to the invention to operate at the fundamental resonant frequency and higher odd harmonics of the combined acoustic wave device and attached cell 300a/300c. (In FIGS. 3A and 3C, symbol 327a/327c represents the mechanical coupling of the attached cell 300a/300c to the acoustic wave device. A similar symbol is used for attached cells throughout the drawings herein). Frequencies corresponding to the resonant frequencies will be passed through, while other frequencies are damped. Since the acoustic wave device acts as a band pass filter of selected resonant frequencies out of the broad spectrum of the white noise generator 301a/301c, the resonant frequencies are shifted by the circuit, in a dynamic fashion, to match the frequency of the combined acoustic wave device plus cell, even as the cell's mechanical properties change in a biological response to a change in conditions.

In addition, the compact nature of a circuit in accordance with an embodiment of the invention allows the circuit to be miniaturized to a high degree, in particular when compared with the use of a network analyzer with a SAW device.

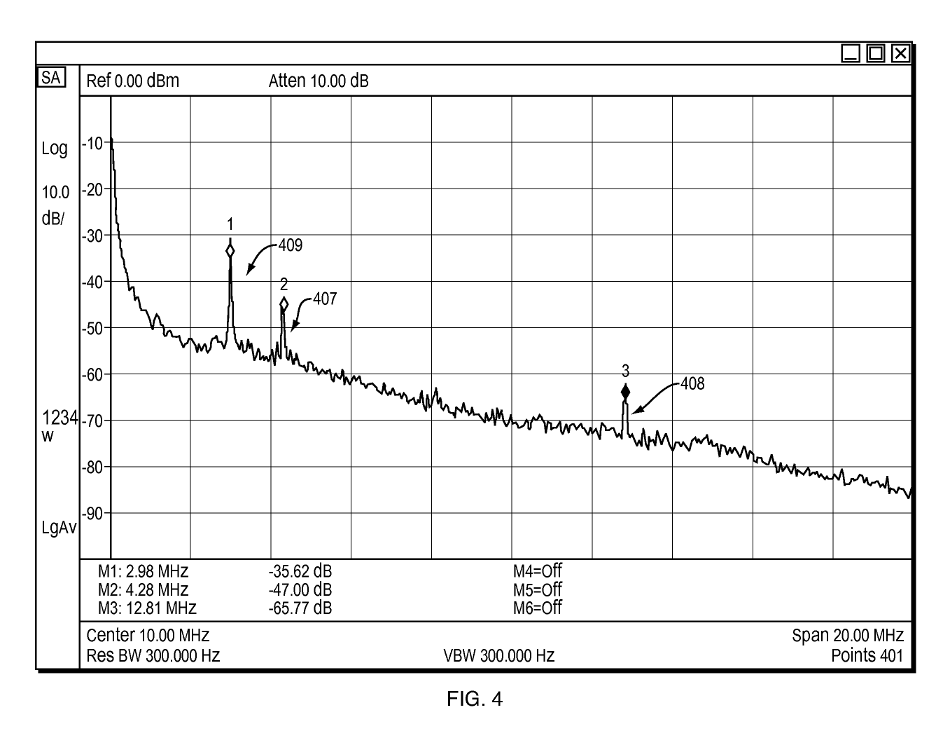

FIG. 4 is a graph of power spectrum data from an active filter using a 4.26 MHz piezo-electric crystal in the feedback loop, in accordance with an embodiment of the invention. The fundamental mode 407 of the crystal is present (4.28 Mhz) as well as the third harmonic 408 of the crystal (12.81 Mhz). The peak 409 observed at 3 MHz is present even when the crystal is removed and is believed to be due to parasitic capacitances in the circuit. Each of the resonant frequencies can be selected out of the other peaks by use of an appropriate band pass filter.

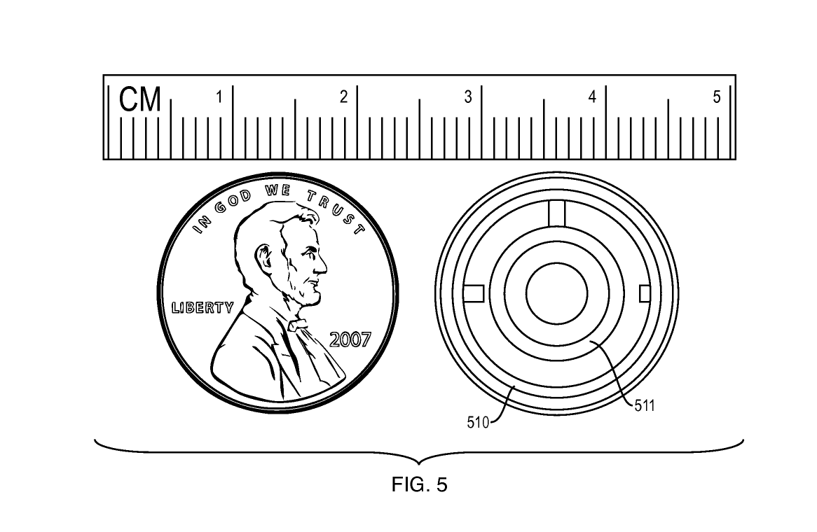

In accordance with an embodiment of the invention, the piezo element itself may consist of a quartz substrate, configured either as a bulk shear mode AW device (also known as a QCM) or a shear mode Surface Acoustic Wave device (SAW). FIG. 5 is a photograph of a QCM-style AW sensor module, in accordance with an embodiment of the invention. The sensor is viewed from the top and compared to a penny for scale. Quartz crystal 510 has a chamber 511 on top of it for holding the support media for the cells. Chamber 511 is bonded to the top of quartz crystal 510. The chamber 511 may be made of a biocompatible polymer. It may be either open at the top for high-throughput automated testing systems, such as a 96-well plate format, or be enclosed with inlet and outlet ports for exchange of media and analyte.

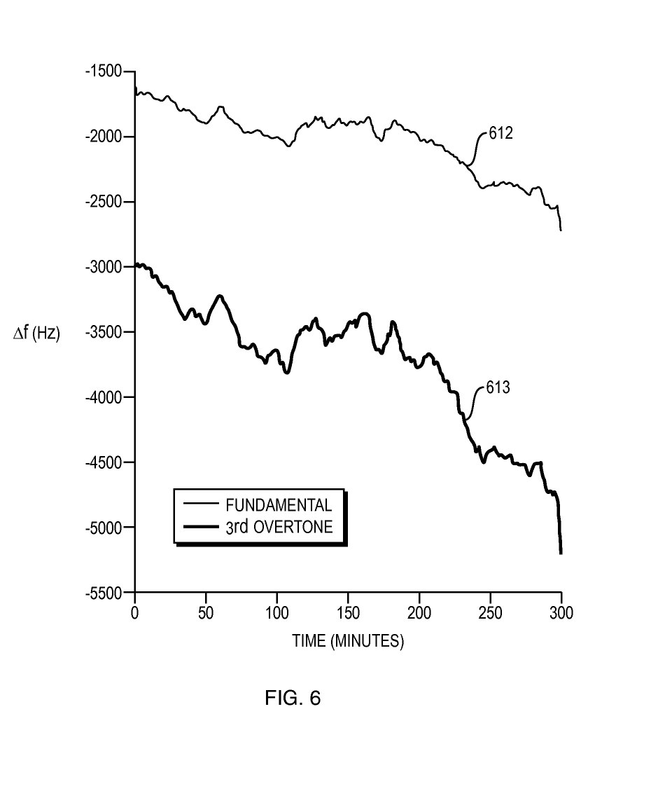

FIG. 6 is a graph showing frequency shift results of an experiment with human mammary epithelial cells treated with growth factor, in accordance with an embodiment of the invention. The frequency shift is shown for the fundamental 612 and third overtone 613 of a 10 MHz QCM having attached the human mammary epithelial cells treated with fibroblast growth factor basic form (bFGF), as described in detail in the below Experimental Demonstration of the Invention. The two signals were collected simultaneously. It can be seen that the third overtone 613 shows a larger frequency shift compared to the fundamental 612 in response to the growth factor.

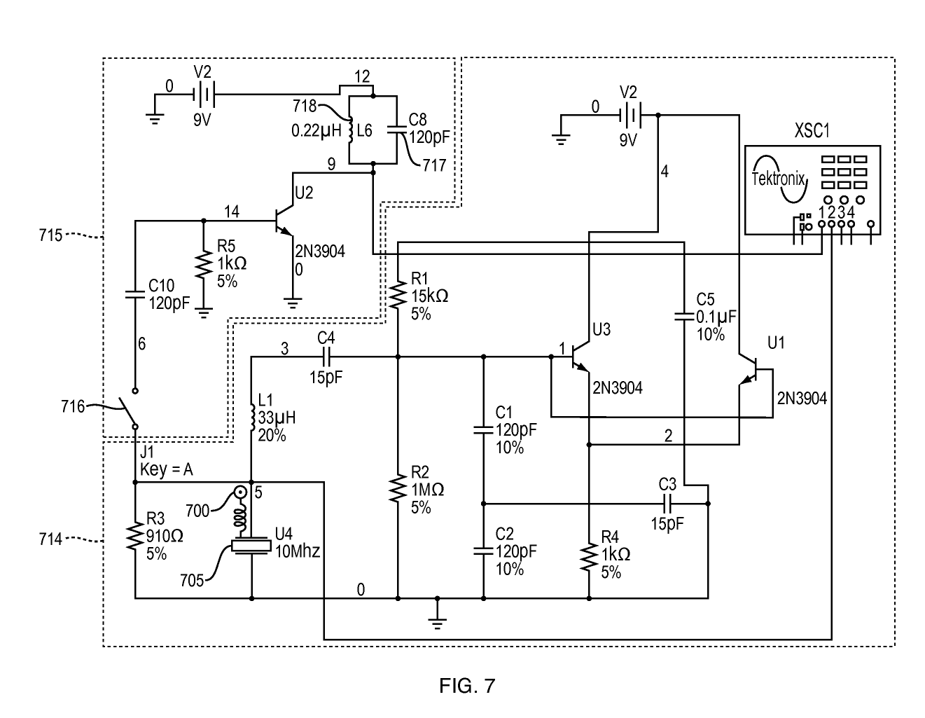

FIG. 7 is a diagram of a sensor circuit using a frequency multiplier, in accordance with another embodiment of the invention. In this embodiment, no white noise generator is used and no band pass filtering occurs. Instead, an acoustic wave device 705 with attached cell 700 is used with an oscillator circuit 714 connected to a multiplier circuit 715. The circuit oscillates at both a fundamental resonant frequency and a higher harmonic, for example a third harmonic, once switch 716 is closed to connect the oscillator circuit 714 to the multiplier circuit 715, which in this case generates the third harmonic. The multiplier circuit 715 may, for example, be implemented using Schottky diodes or as a frequency multiplier circuit with a bipolar junction transistor in a Class C configuration. To add to the flexibility of the circuitry, the capacitor 717 and inductor 718 of the multiplier circuit 715 can be replaced with an array (not shown) which may be digitally selected via an analog switch array (not shown). By selecting the appropriate series of capacitors 717 and inductors 718 via the switch array, the L-C values can be chosen to select a specific frequency band. This frequency can be changed by altering the specific inductors 718 and capacitors 717 that are connected to the rest of the circuit by way of the switch 716. Selected appropriately, the range of frequencies can cover the desired frequency range for the fundamental oscillation mode and a number of higher harmonic modes. This configuration allows for a single circuit to handle multiple harmonics by being dynamically switched between them. It also allows for dynamic tuning of the circuit to optimize the oscillation for any given harmonic. If a crystal that is nominally 10 MHz, but is oscillating at 8 MHz under liquid and cellular loading, the 10 MHz tuning of the L-C circuit is no longer optimal and there may be a decreased amplitude of oscillation solely due to this effect, having nothing to do with the crystal's condition. Since the frequency may be sampled either locally by a microcontroller or through an interface with a PC, software algorithms can be employed to calculate the best tuning frequency for the L-C network and to digitally select the appropriate values from the capacitors 717 and inductors 718 to attain this.

In one embodiment, the invention is a method of sensing a response of a living cell or virus to a change in conditions. An essentially constant external electromotive force is applied that causes oscillation of an acoustic wave device, such as a crystal or piezo-element (for example, 305a of FIG. 3A) having attached a living cell or virus, wherein the acoustic wave device and cell or virus are oscillating at essentially constant amplitude and frequency under steady state conditions. For example, in FIG. 3A, the essentially constant external electromotive force may be the voltage across component 305a in the circuit of FIG. 3A. Herein, an “essentially constant external electromotive force” means an externally-applied electromotive force that results in oscillation of component 305a at essentially constant frequency and amplitude when both the cell and component 305a are at steady state. As used herein, a “living cell” is any cell containing genetic material that can exhibit new gene expression as a result of environmental stimuli. However, it should be understood that, as used herein, a response of the living cell or virus does not necessarily have to be a consequence of any new genetic expression. A combined oscillating system, including the acoustic wave device 305a and the attached living cell or virus exhibits a fundamental frequency and at least one harmonic frequency of the combined oscillating system under steady state conditions. The living cell or virus is exposed to a change in an environmental condition while oscillating the combined oscillating system under the essentially constant external electromotive force. Herein, exposing the living cell or virus to a “change in environmental condition” means exposing the cell or virus to any change in the environment of the cell or virus that may potentially prompt a biological response by the cell, for example exposing the cell or virus to a drug candidate for the living cell or virus, a toxin for the living cell or virus, a carcinogen for the living cell or virus, a pathogen for the living cell or virus, a virus, a bacterium, a fungus, a protozoa, a gas, and/or a depletion of oxygen. A response of the living cell or virus to the change in environmental condition will be indicated by a change in at least one of frequency and amplitude of the oscillation of at least one harmonic frequency of the combined oscillating system.

In a specific embodiment of the invention, the essentially constant external electromotive force is applied by band-pass filtering an electrical driving signal through the acoustic wave device. For example, in FIG. 3A, the electromotive force produced by a white noise generator 301a is band-pass filtered through component 305a by applying an electrical driving signal across component 305a. The electrical driving signal may be band-pass filtered by passing the electrical driving signal through an amplifier circuit that includes the acoustic wave device as a feedback element of the amplifier circuit, as shown, for example, in FIG. 3A, in which component 305a is a feedback element of the amplifier circuit that includes amplifier 304a. Alternatively, the electrical driving signal may be band-pass filtered by passing the electrical driving signal through a passive circuit that includes an amplifier and the acoustic wave device, as shown, for example, in FIG. 3C, in which component 305c is part of a passive circuit that includes the amplifier 304c and component 305c. Under the essentially constant external electromotive force, the combined oscillating system may exhibit a fundamental frequency and a plurality of harmonic frequencies of the combined oscillating system. For example, more than one odd harmonic frequencies may be exhibited in addition to the fundamental frequency, such as the fundamental, third, fifth and seventh harmonics of the fundamental frequency.

The method of the invention, in another specific embodiment, may include the step of generating an electrical signal in an electrical circuit electrically coupled to the acoustic wave device, such as in the electrical circuits of FIGS. 3A and 3C and/or the frequency counter and amplitude measurement block 203 of FIG. 2. The electrical signal may include a component indicating a change in at least one of frequency and amplitude of the oscillation for at least one harmonic frequency of the combined oscillating system, over at least a portion of a time span during which the living cell or virus responds to the change in environmental condition. For instance, in FIG. 6, an electrical signal is shown that includes a component indicating a change in frequency of the third harmonic 613 of a combined oscillating system that includes the acoustic wave device and a human mammary epithelial cell, over the time span during which the human mammary epithelial cell responds to a growth factor.

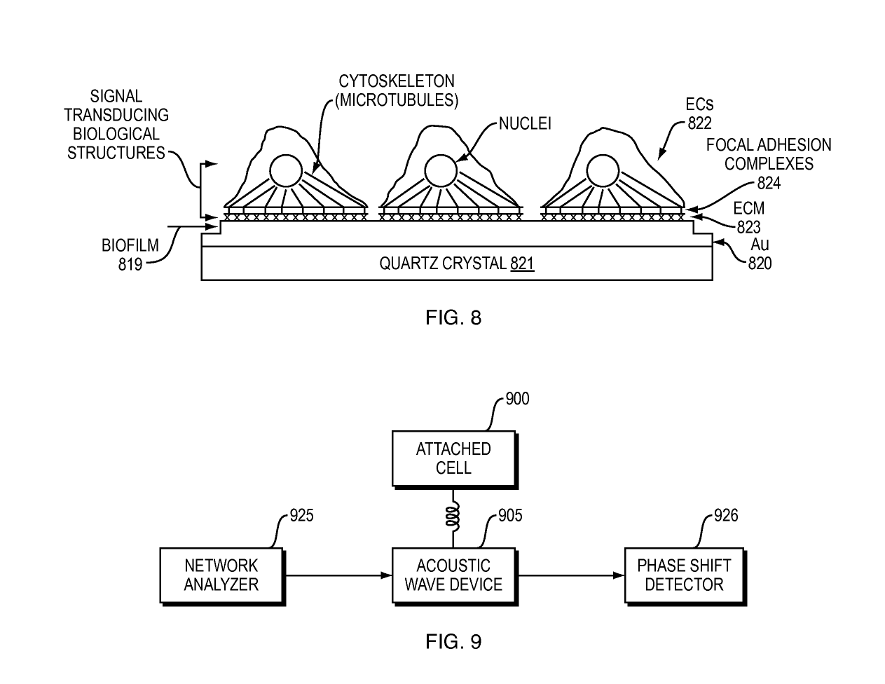

The change in at least one of frequency and amplitude of the oscillation at the fundamental and harmonic frequencies of the combined oscillating system may be responsive to a change in a subcellular structure of the cell or virus. For instance, if the microtubules or another subcellular structure of the cell change in shape or location, the change in frequency or amplitude may respond to that change. The acoustic wave device may include a surface acoustic wave device, a bulk acoustic wave device, a quartz crystal microbalance device, a Love wave device, a torsional resonator, and/or a piezoelectric acoustic wave device under a damping mechanical load of a fluid immersing the living cell or virus. The acoustic wave device may include a selective substrate film disposed onto a surface of the acoustic wave device. The living cell or virus may be attached to the selective substrate film by a cell-surface molecule bound to a binding site on the selective substrate film. For example, a selective substrate film set forth in U.S. Pat. No. 7,566,531 B2 of Marx et al., the entire disclosure of which is hereby incorporated herein by reference, may be used, such as a polymer of one or more of: phenolic compounds, aniline derivatives, tyrosines, tyrosine derivatives, a tyrosine-containing peptide, or a combination thereof FIG. 8 is a diagram of a biosensor using such a selective substrate film, in accordance with an embodiment of the invention. FIG. 8 shows the signal transduction region of a whole cell QCM biosensor (although other types of acoustic wave devices discussed herein may be used), in which a layer or film 819 of a selective substrate is applied to the surface of a conducting element 820 (such as gold (Au)) of the QCM, which is on top of the quartz crystal 821. The adherent cells 822 (here, endothelial cells or EC's) stably adhere to the selective substrate film or layer 819 and reach a steady state. Initially, the cells adhere to binding sites in the selective substrate film, but later deposit an extracellular matrix (ECM) 823 and spread across the surface of the selective substrate film, and attach themselves more firmly by formation of specific complexes such as focal adhesion complexes (FAC's) 824 to which the cytoskeleton of the cells is coupled.

In addition, the living cells may adhere to the surface of the acoustic wave device, for example in the fashion described in U.S. Patent App. Pub. No. 2003/0008335 A1 of Marx et al., the entire disclosure of which is hereby incorporated herein by reference.

In another embodiment according to the invention, in place of using a living cell or virus described herein, a molecularly-imprinted polymer may be used, such that a response of the molecularly-imprinted polymer to a change in conditions may be sensed. For example, the molecularly-imprinted polymer may be mounted on an acoustic wave device, for example by (but not limited to), having the molecularly-imprinted polymer be mounted on a selective substrate film. The molecularly-imprinted polymer is exposed to a change in an environmental condition, whereby a response of the molecularly-imprinted polymer to the change in environmental condition will be indicated by a change in at least one of frequency and amplitude of the oscillation of at least one harmonic frequency of the combined oscillating system. In particular, the output of a white noise generator may be band pass filtered through the acoustic wave device in a similar fashion to that described above. The molecularly-imprinted polymer may be designed, when synthesized, to recognize a specific analyte. For example, the molecularly-imprinted polymer may recognize a living cell or other analyte when the molecularly-imprinted polymer is exposed to the living cell or other analyte. For example, a molecularly-imprinted polymer may be an amino acid detecting film. In one example, the amino acid detecting film may be a film that specifically detects L-Glutamic acid, as described in B. Deore, Z. Chen and T. Nagaoka (2000), Potential-Induced Enantioselective Uptake of Amino Acid into Molecularly-Imprinted Overoxidized Polypyrrole, Analytical Chemistry, 72, 3989-3994, the entire disclosure of which is hereby incorporated herein by reference. A specific example of such a molecularly-imprinted polymer is an overoxidized polypyrrole. In another embodiment, the molecularly-imprinted polymer may be sensitive to a drug or other chemical. In one example, a molecularly-imprinted polymer may be sensitive to morphine, as described in D. Kriz, K. Mosbach, Anal. Chim Acta 1995, 300, 71, the entire disclosure of which is hereby incorporated herein by reference. A specific example of such a molecularly-imprinted polymer is a polymethacrylate film imprinted with morphine.

FIG. 9 is a block diagram of a sensor configured to determine a change in complex impedance, in accordance with an embodiment of the invention. In this embodiment, a response of the living cell or virus to the change in environmental condition will be indicated by a change in a complex impedance of the combined oscillating system, for example, a change in impedance due to inductive and/or capacitive impedance rather than merely resistive impedance. A network analyzer 925 is connected with the acoustic wave device 905, such as a SAW device, with attached cell 900, thereby inducing the change in complex impedance. A phase shift detector 926 permits obtaining the change in complex impedance of the combined oscillating system.

In accordance with an embodiment of the invention, the living cell or virus may be, at least in part, a living mammalian cell, a living non-mammalian cell, a bacteria cell, an archaea cell, a genetically modified cell, and/or a genetically-engineered cell.

The living cell or virus employed in accordance with an embodiment of the invention may include a mixture of more than one type of living cell that mimics a response of at least a portion of a tissue or an organ. For example, the mixture of more than one type of living cell may include at least one member of the group consisting of: a lung epithelial cell; a lung endothelial cell; a lung fibroblast; a lung macrophage; a gastrointestinal epithelial cell; a gastrointestinal endothelial cell; a gastrointestinal goblet cell; a gastrointestinal fibroblast; an astroglial cell; a neuron; a brain endothelial cell; a brain fibroblast; a retinal pigmented epithelial cell; a retinal neuron; a retinal ciliary cell; a corneal epithelial cell; a squamous epithelial cell; a keratinocyte; a melanocyte; a fibroblast; a dermal endothelial cell; and a smooth muscle cell. For example, the foregoing mixtures may be used to mimic the population of cells that co-exist in the lung, to simulate the cells that co-exist and communicate in the gastrointestinal tract, to simulate the brain and/or blood brain barrier, to simulate the eye, and/or to simulate the skin. It will be appreciated, however, that a variety of other different possible cells may be used, including any cell type with a nucleus capable of responding to an external stimulus, and other cells, including cells that do not have a nucleus. The mixture of more than one type of living cell may include at least one adherent nucleated cell derived from at least one member of the group consisting of: endoderm, mesoderm and ectoderm. Further, the mixture of more than one type of living cell may include at least one stem cell derived from at least one member of the group consisting of: endoderm, mesoderm and ectoderm.

The living cell may be, at least in part, a eukaryotic cell, a mutant cell, a diseased cell, a tumor cell (for example a human tumor cell, such as a breast cancer cell), an endothelial cell and/or an epithelial cell. Further, the living cell or virus may be, at least in part, at least one member of the group consisting of: a virally-infected cell; a bacterially-infected cell; a fungally-infected cell; a protozoa-infected cell; and an amoebic-infected cell; including human cells infected with the foregoing. Further, the living cell may be, at least in part, a cell infected with a resistant strain of an organism, including a human cell so infected.

In further related embodiments, exposing the living cell or virus to a change in environmental condition may include exposing the living cell or virus to a drug candidate for the living cell or virus. Further, exposing the living cell or virus to a change in environmental condition may include exposing the living cell or virus to at least one member of the group consisting of: a toxin for the living cell or virus; a carcinogen for the living cell or virus; and a pathogen for the living cell or virus. Exposing the living cell or virus to a change in environmental condition may include exposing the living cell or virus to at least one member of the group consisting of: a virus, a bacterium, a fungus and a protozoa; and/or exposing the living cell or virus to a gas; and/or exposing the living cell or virus to a depletion of oxygen.

It should be appreciated that, although both the acoustic wave device and the attached living cell or virus are oscillating, it is only the acoustic wave device that is directly connected to the associated electronics. Nevertheless, the oscillations of the acoustic wave device reflect the biological response of the cell or virus to a change in environmental conditions, since the cell or virus is mechanically attached to the acoustic wave device.

In some embodiments, the invention can be used in public places for environment monitoring. For example, it can have associated air and temperature regulation, or can be used without air and temperature regulation. In a specific embodiment, the device may be sold in two parts: the electronics and mount may be a one time purchase and serve as a platform for a multitude of consumables containing the living cells.

A frequency response of a cell generated by the method of the invention may show a number of peaks, each corresponding to a specific cellular structure. By utilizing drugs that target specific structures within a cell, such as the microtubules, the frequency response may be analyzed for signature patterns that correlate with the changes in that structural element.

### References:

1. Ahuja A, James DL, Narayan R. Dynamic behavior of ultra-thin polymer films deposited on surface acoustical wave devices. Sensors and Actuators A: Physical. 1999; 72(3):234-41.

2. Gee W A, Ritalahti K M, Hunt W D, Lomer F E. QCM viscometer for bioremediation and microbial activity monitoring. Sensors Journal, IEEE. 2003; 3(3):304-9.

3. Nwankwo E, Durning C J. Fluid property investigation by impedance characterization of quartz crystal resonators: Part II: Parasitic effects, viscoelastic fluids. Sensors and Actuators A: Physical. 1999;72(3):195-202.

4. Calabrese G S, Wohltjen H, Roy M K. Surface acoustic wave devices as chemical sensors in liquids. Evidence disputing the importance of Rayleigh wave propagation. Analytical Chemistry. 1987; 59(6):833-7.

5. Ali Z. Acoustic Wave Mass Sensors. Journal of Thermal Analysis and calorimetry. 1999; 55(2):397-412.

6. Josse F, Bender F, Cernosek R W. Guided Shear Horizontal Surface Acoustic Wave Sensors for Chemical and Biochemical Detection in Liquids. Analytical Chemistry. 2001;73(24):5937-44.

7. JUTS P C, Bakken G A, McClelland H E. Computational Methods for the Analysis of Chemical Sensor Array Data from Volatile Analytes. Chemical Reviews. 2000; 100(7):2649-78.

8. Rocha-Gaso Ma-I, March-Iborra C, Montoya-Baides An, Arnau-Vives A. Surface Generated Acoustic Wave Biosensors for the Detection of Pathogens: A Review. Sensors. 2009; 9(7):5740-69.

9. Weigel R, Morgan D P, Owens J M, Ballato A, Lakin K M, Hashimoto K, et al. Microwave acoustic materials, devices, and applications. Microwave Theory and Techniques, IEEE Transactions on. 2002; 50(3):738-49.

10. Ohring M. The materials science of thin films: deposition and structure: Academic Press; 2002.

11. B. Deore, Z. Chen and T. Nagaoka (2000), Potential-Induced Enantioselective Uptake of Amino Acid into Molecularly-Imprinted Overoxidized Polypyrrole, Analytical Chemistry, 72, 3989-3994.

12. D. Kriz, K. Mosbach, Anal. Chim Acta 1995, 300, 71.

13. E. Fukada, History and Recent Progress in Piezoelectric Polymers, IEEE Transactions on Ultrasonics, Ferroelectrics, and Frequency Control, Vol. 47, No. 6, November 2000.

The teachings of all patents, published applications and references cited herein are incorporated by reference in their entirety.

The following represents an example of the invention and is in no way limiting.

### Experimental of Increased Sensitivity of Third Harmonic in QCM

### 1. Demonstration of Increased Sensitivity of Third Harmonic in QCM

An AT cut 0.538″ diameter quartz disk with 100 nm thick gold electrodes was used for the piezoelectric medium in this experiment. A polydimethylsiloxane (PDMS) tube with an outer diameter of 0.4″ and an inner diameter of 0.3″ and 0.3″ high was attached to one side of the quartz disk for use as a reservoir for cell media. The quartz crystal was mounted in a HC35/U holder with silver epoxy bonding between the electrodes and the gold electrodes on the crystal.

The oscillator circuitry is a Colpitts oscillator, shown in FIG. 7, modified to enable the quartz to undergo shear wave oscillations under a liquid load. The thickness of the crystal is such that an unloaded crystal oscillating in air would operate at 10 MHz. The Colpitts oscillator was tuned to only amplify frequencies in the range of 10 MHz. A second class C amplifier operating as a frequency multiplier was connected to the ungrounded electrode of the quartz crystal. The multiplier was tuned to capture the crystal's third harmonic oscillations. The output of the multiplier and the voltage at the crystal were connected to the inputs of a two channel oscilloscope. The waveforms were digitally captured using Labview software and passed through a fast Fourier transform. The frequency of the signals were captured and recorded at one minute intervals, prior to and following addition of cells to the crystal surface.

Human Mammary Epithelial Cells (HMECs) were purchased from Lonza (Basel, Switzerland) and grown according to the manufacturer's instruction in MEGM media and bullet kit at 37° C. in a humidity controlled incubator with 5% CO₂. The HMEC cells were grown in 25 cm² rectangular canted neck cell culture flasks with vented caps (Corning Life Science, Lowell, Mass.), and re-fed every three days and passaged at least once a week as stock cultures.

Basic human Fibroblast Growth Factor (bFGF) was obtained from Sigma (St. Louis, Mo.) and was reconstituted in 50 mM Tris-HC1 pH7.5 (at 50 μg/m1). bFGF is found in basement membranes and sub-endothelial extracellular matrix. bFGF is induced early in the development of the embryo where it is necessary (but not sufficient) for pluripotency and self-renewal of embryonic stem cells (ESC) and lineage defined stem cells; wherein it functions to support proliferation and inhibit differentiation. When added to HMECs, bFGF induces a proliferative response following a lag time of about 21 hr as measured by an increase in thymidine incorporation indicative of synthesis of DNA. An increase in cell number requires 48-72 hrs to measure a response to a mitogen such as fibroblast growth factor in normal, unsynchronized cell culture populations.

Prior to cell plating, the gold QCM surface was cleaned before assembly in the well holder. After sterilization and washing with water and Phosphate Buffered Saline (PBS), the QCM device was placed within a large petri dish, filled with distilled water, and covered with a piece of PDMS, then placed inside a 37 degree temperature-regulated cell incubator with atmosphere controlled at 5% CO₂ for 24 hr prior to the cell plating and attachment step. Then a 200 μl of medium containing 20,000 HMEC cells was added within the well onto the gold QCM surface and allowed to attach to the gold QCM surface in the incubator. Then the QCM was removed and placed into ambient air and room temperature and the QCM electrode was plugged into a Colpitts oscillator. At this point, the QCM device collected frequency data at the fundamental and third overtone frequencies of the crystal. Frequencies were collected for a total of 27 hr prior to addition of bFGF to a final concentration of 12.5 ng/ml (see FIG. 6). In FIG. 6, where the x-axis is labeled 0 min. corresponds to 27 hr into the experimental collection of frequency data where the cells are attached to the surface, at which time the bFGF was added. Following bFGF addition, frequency data continued to be collected for an additional 300 min., as shown in FIG. 6 where these frequency data for the fundamental and third overtone frequencies following bFGF addition are displayed. The frequencies correspond to an early HMEC cell response to bFGF, prior to any manifestation by doubling of cell number, mitotic figures or DNA synthesis, but may be related to membrane receptor tyrosine kinase pathways being activated, since the growth factor has been shown to bind and activate its specific growth factor receptor within 8 minutes.

### 2. Sensing of Cellular Response Using a Surface Acoustic Wave Device

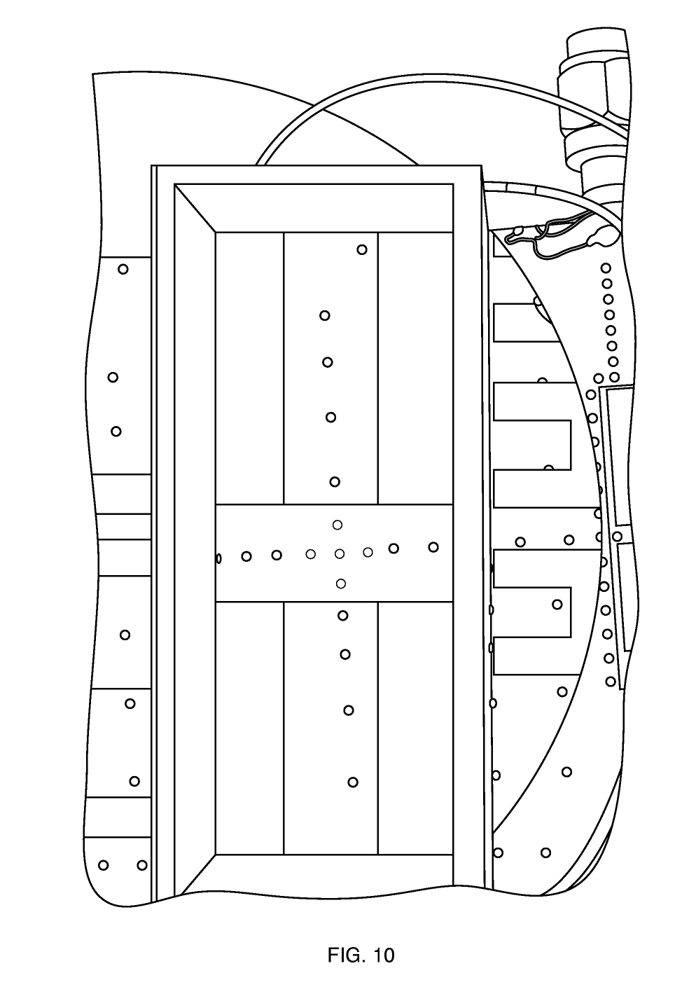

An AT cut quartz wafer was prepared with electrodes for a surface acoustic wave (SAW) patterned on one side. The transmitting and receiving electrodes were composed of interdigitated (IDT) electrodes fabricated by lift off of gold deposited by electron beam evaporation. The IDTs were composed of 120 pairs of electrode fingers. Each finger was 7960×40 μm with a center-to-center spacing between adjacent fingers of 80 μm, as shown in FIG. 10. This spacing corresponds to the wavelength of a SAW of 21 MHz. The sensing area between the IDTs was 20×8 mm. A 2 μm thick layer of poly(methylmethacrylate) (PMMA) was spin coated onto the surface of the SAW to serve as an insulating layer for the IDTs as well as a waveguide for the Love waves generated by the transmitting IDT.

The SAW was connected to an Agilent E5061B network analyzer, set up for transmission measurements (s12). Before liquid was added, the resonant frequency of 21 MHz was confirmed. Using Labview software, the resonant frequency and attenuation of the SAW was measured once per second during an experiment. A drop of cell media containing cells was placed in the sensing area allowing cells to attach, as described below.

The Human Microvascular Endothelial Cells derived from Lung (HMVEC-L) cell line was obtained from Lonza (Basel, Switzerland) and stock cultures were established and grown in media recommended by the manufacturers, at 37° C. in a humidity controlled incubator with 5% CO₂, unless otherwise noted. The media used was EBMTM-2, (CC-3156) (Lonza Basel, Switzerland) with EGMTM-2-MV BulletKit™ (CC-3202) (Lonza Basel, Switzerland) containing hEGF, Hydrocortisone, GA-1000 (Gentamicin, Amphotericin-B), VEGE, hFGF-B, R3-IGF-1 and 5% FBS. The HMVEC-L cells were grown in 25 cm² rectangular canted neck cell culture flasks with vented caps (Corning Life Science, Lowell, Mass.), and re-fed every three days and passaged once a week using a 0.05% solution of trypsin:EGTA (Invitrogen, Carlsbad, Calif.) as stock cultures.

Two different treatments of the cells on the SAW device were carried out in separate experiments. These were final concentrations of 50 mM sodium azide, and 0.05% solution of trypsin:EGTA (Invitrogen, Carlsbad, Calif.). Sodium azide (S8032) was purchased from Sigma (St. Louis, Mo.). A stock solution of 1M sodium azide was prepared by dissolving 130 mg of sodium azide in 2 m1 pH 7 PBS (0.05 M). The 50 mM sodium azide was made by further dilution of the stock solutions with pH 7 PBS. All the solutions were stored at 4° C. in a refrigerator. Prior to cell plating onto the SAW device, the SAW device was cleaned by rinsing with distilled water and then UV sterilized for 30 min After sterilization and further washing with water and Phosphate Buffered Saline (PBS), 50 μl of medium containing 20,000 HMVEC-L cells was added to the sensing area of the SAW device, which had been previously placed within a 150 mm diameter Petri dish. Smaller Petri dishes filled with reservoirs of media were placed around the central SAW device, in order to provide additional surface area for evaporation of water. The purpose of this arrangement, large volume and area reservoirs of iso-osmotic pressure media in the Petri dishes, was to prevent evaporative loss of water from the small volume of media covering the cells in the sensing area of the SAW device. The SAW device in the large covered Petri dish was located on the bench top in ambient air. The Petri dish cover was removed and cells were plated in media onto the sensing area surface of the SAW device and the HMVEC-L cells in the covered Petri dish were allowed to attach to the sensing area SAW device surface for 18 hr. Then, the media was changed with 50 μl fresh medium. Following a 2 hr interval post-media change, either the trypsin removal experiment or the sodium azide treatment experiment were carried out as described below.

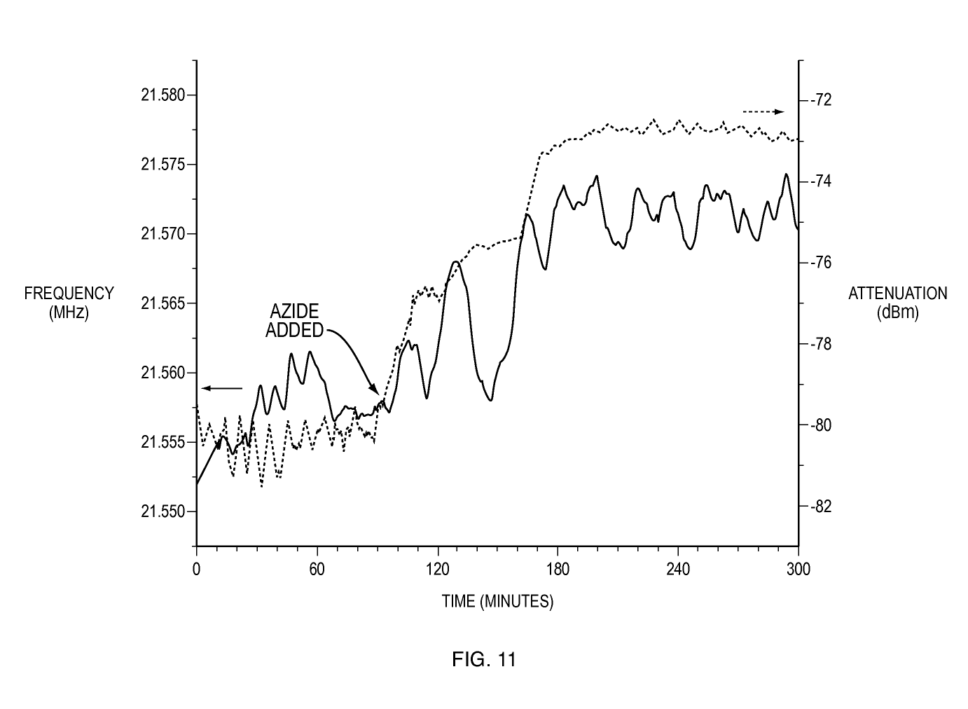

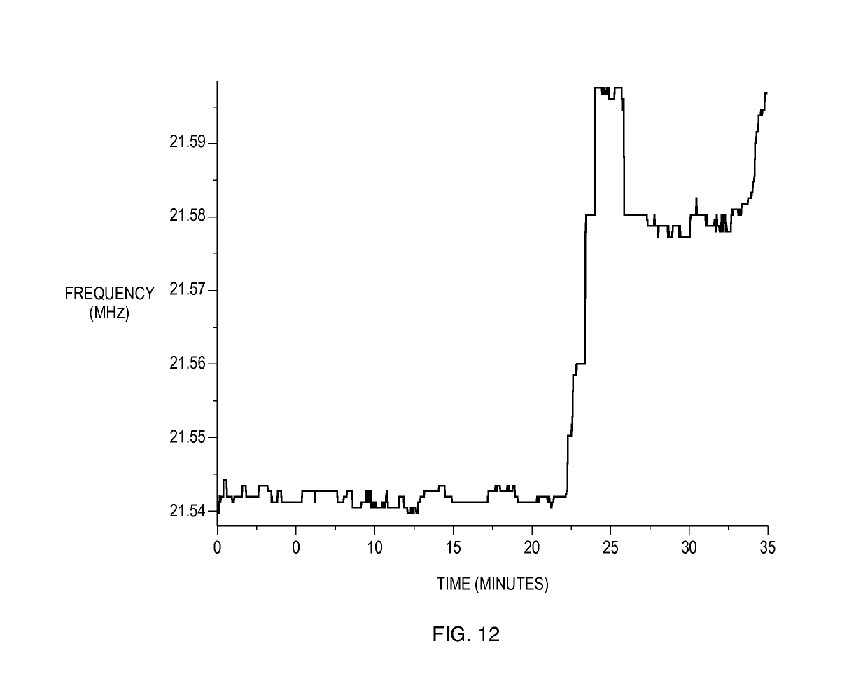

For the sodium azide experiment, a volume of sodium azide was added from the stock solution to achieve a 50 mM final concentration in the media volume containing the 20,000 HMVEC-L cells on the SAW device sensing area. The frequency data was recorded prior to this addition and for the next 12 hr post-addition. At the end of the experiment, the number of cells adhering to the SAW surface was determined by multiple trypsinizations (with 0.05% trypsin with EDTA (Invitrogen, Carlsbad, Calif.)) and electronic cell counting by cellometer which also identifies live versus dead cells by the inclusion of a viability dye in the counting media (Nexcelom Bioscience LLC., Lawrence, Mass.). Trypsin treatment removes cells and cellular protein from the crystal surface and the crystal oscillation frequency increases significantly to pre-cell addition levels. In FIG. 11 we show the measured frequency prior to sodium azide addition to 50 mM for 20,000 HMVEC-L cells on the SAW device sensing area, as well as the response following addition for 4 hr. These data show an increase in the frequency of about 17 kHz as a result of sodium azide addition. This increase is believed to be due to disruption of the mitochondria structures, including mitochondrial membrane depolarization within the cells. In FIG. 11, we also display the Attenuation values from the SAW device. As a result of azide addition to 50 mM, the Attenuation values undergo a significant change by approximately 8 dBm. For the second experiment, the tyrosine release experiment on the same number of 20,000 HMVEC-L cells on the SAW device sensing area, we measured an abrupt increase in frequency of about 60 kHz, as shown in FIG. 12. This frequency increase results from the trypsin catalytic activity releasing these cells from the SAW surface.

In the experiments we performed, the SAW device with 20,000 HMVEC-L cells added to the sensing area surface of the SAW device for the 50 mM sodium azide experiment and the trypsinization experiment were compared for their sensitivities to equivalent experiments performed previously with the same number of HMVEC-L cells in identical media in a commercial quartz crystal microbalance (QCM) device operated at 10 MHz fundamental frequency. The directions of the frequency shifts were similar for both devices but the magnitudes were different. Experimental frequency shifts with the SAW device normalized to both its 21 MHz operating frequency and the total number of live cells firmly attached to the SAW surface at the end of the experiment as determined by the number of cells trypsinized from the surface that exclude the viability dye and are electronically counted, yields sensitivities of 5.6×10⁻⁸/cell for the sodium azide experiment and 3.75×10⁻⁷/cell for the Trypsin enzymatic removal experiment. This compares to equivalent sensitivities we have determined from prior experiments we carried out on the same number of HMVEC-L cells using the QCM at 10 MHz. These were 4.1×10⁻⁹/cell for the 50 mM azide experiment and 1×10⁻⁸/cell for the trypsinization experiment. Comparing these different device sensitivities as the ratio of sensitivities, there is a larger magnitude response to each of these changes in the cells on the sensing area of the SAW device compared to the QCM used with the embodiments of FIGS. 6 and 7. The trypsinization cell release experiment was ˜38-fold more sensitive than the QCM device at detecting this perturbation at the device surface. For the 50 mM sodium azide experiment, the SAW device was ˜14-fold more sensitive compared to the QCM device.

While this invention has been particularly shown and described with references to example embodiments thereof, it will be understood by those skilled in the art that various changes in form and details may be made therein without departing from the scope of the invention encompassed by the appended claims.

1. A method of sensing a response of a living cell or virus to a change in conditions, the method comprising the steps of:
a) applying an essentially constant external electromotive force that causes oscillation of an acoustic wave device at essentially constant amplitude and frequency under steady state conditions, the acoustic wave device having attached at least one living cell or virus, whereby a combined oscillating system including the acoustic wave device and the living cell or virus exhibits a fundamental frequency and at least one harmonic frequency of the combined oscillating system; and
b) exposing the living cell or virus to a change in an environmental condition while oscillating the combined oscillating system under the essentially constant external electromotive force, whereby a response of the living cell or virus to the change in environmental condition will be indicated by a change in at least one of frequency and amplitude of the oscillation of at least one harmonic frequency of the combined oscillating system.

2. A method according to claim 1, wherein the essentially constant external electromotive force is applied by band pass filtering an electrical driving signal through the acoustic wave device.

3. A method according to claim 2, wherein the output of a white noise generator is band pass filtered through the acoustic wave device.

4. A method according to claim 2, wherein the electrical driving signal is band-pass filtered by passing the electrical driving signal through an amplifier circuit that includes the acoustic wave device as a feedback element of the amplifier circuit.

5. A method according to claim 2, wherein the electrical driving signal is band-pass filtered by passing the electrical driving signal through a passive circuit that includes an amplifier and the acoustic wave device.

6. A method according to claim 1, wherein, under the essentially constant external electromotive force, the combined oscillating system exhibits a fundamental frequency and a plurality of harmonic frequencies of the combined oscillating system.

7. A method according to claim 1, further including the step of generating an electrical signal in an electrical circuit electrically coupled to the acoustic wave device, the electrical signal including a component indicating a change in at least one of frequency and amplitude of the oscillation for at least one harmonic frequency of the combined oscillating system, over at least a portion of a time span during which the living cell or virus responds to the change in environmental condition.

8. A method according to claim 1, wherein the change in at least one of frequency and amplitude of the oscillation at the fundamental and harmonic frequencies of the combined oscillating system is responsive to a change in a subcellular structure of the cell or virus.

9. A method according to claim 1, wherein the acoustic wave device includes a surface acoustic wave device.

10. A method according to claim 1, wherein the acoustic wave device includes at least one member of the group consisting of: a bulk acoustic wave device, a quartz crystal microbalance device, a Love wave device and a torsional resonator.

11. (canceled)

12. A method according to claim 1, wherein the acoustic wave device includes a piezoelectric acoustic wave device under a damping mechanical load of a fluid immersing the living cell or virus.

13. and 14. (canceled)

15. A method according to claim 1, wherein the acoustic wave device includes a selective substrate film disposed onto a surface of the acoustic wave device, the living cell or virus being attached to the selective substrate film by a cell-surface molecule bound to a binding site on the selective substrate film.

16. through 44. (canceled)

45. An apparatus for sensing a response of a living cell or virus to a change in conditions, the apparatus comprising:
a) an acoustic wave device; and
b) an electromotive drive connected to the acoustic wave device that causes oscillation of the acoustic wave device at a fundamental and at least one harmonic frequency, the electromotive drive including a signal generator electrically coupled to provide an electrical driving signal that is band pass filtered through the acoustic wave device.

46. An apparatus according to claim 45, wherein the signal generator includes a white noise generator.

47. An apparatus according to claim 45, further including an amplifier circuit, wherein the signal generator is electrically coupled to pass the electrical driving signal into the amplifier circuit, and whereby the acoustic wave device operates as a feedback element in the amplifier circuit to band pass filter the electrical driving signal.

48. An apparatus according to claim 45, further including a passive circuit including an amplifier and the acoustic wave device, wherein the signal generator is electrically coupled to pass the electrical driving signal into the amplifier circuit, and whereby the acoustic wave device operates to band pass filter the electrical driving signal.

49. An apparatus according to claim 45, wherein the acoustic wave device includes at least one member of the group consisting of: a surface acoustic wave device, a bulk acoustic wave device, a quartz crystal microbalance device, a Love wave device and a torsional resonator.

50. through 53. (canceled)

54. An apparatus according to claim 45, the apparatus including at least one living cell or virus attached to the acoustic wave device, wherein the electromotive drive causes oscillation of a combined oscillating system including the acoustic wave device and the living cell or virus at a fundamental and at least one harmonic frequency of the combined oscillating system.

55. An apparatus according to claim 54, wherein the acoustic wave device includes a piezoelectric acoustic wave device under the damping mechanical load of a fluid immersing the living cell or virus.

56. An apparatus according to claim 54, wherein the acoustic wave device includes a selective substrate film disposed onto a surface of the acoustic wave device, the living cell or virus being attached to the selective substrate film by a cell-surface molecule bound to a binding site on the selective substrate film.

57. through 87. (canceled)
